(19) United States
(12) Patent Application Publication
(10) Pub. No.: US 2026/0198979 A1
(43) Pub. Date: Jul. 16, 2026

(54) BONE CEMENT MIXING SYSTEM WITH INTER-CHAMBER VALVE

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 19/444,985

(22) Filed: Jan. 9, 2026

(30) Foreign Application Priority Data
Jan. 15, 2025 (EP) 25152024.3

(51) Int. Cl. A61B 17/88; A61B 17/00

(52) U.S. Cl. CPC A61B 17/8833; A61B 2017/00544; A61B 2017/00964; A61B 2017/8838

(57) ABSTRACT

A device for producing a bone cement including a storage container for receiving a sealed vessel containing monomer liquid, and a cartridge for mixing bone cement, wherein the cartridge comprises a first chamber for receiving monomer liquid from the storage container and a second chamber for receiving bone cement powder, wherein the cartridge further comprises a valve which fluidically connects first chamber to second chamber, wherein the device is designed and configured, in a first step, to release monomer liquid from sealed vessel within the storage container and to deliver it to first chamber of cartridge, in a second step, to keep monomer liquid within the first chamber, wherein contact of the monomer liquid with the second chamber of the cartridge is avoided, and, in a third step, to deliver monomer liquid from the first chamber of the cartridge into the second chamber of the cartridge.

### CROSS-REFERENCE TO RELATED APPLICATION

This application claims priority pursuant to 35 U.S.C. 119(a) to European Patent Application No. 25152024.3, filed January 15, 2025, which application is incorporated herein by reference in its entirety.

### FIELD OF THE INVENTION

The present invention relates to the field of medical engineering, in particular to devices for producing a PMMA bone cement and for storing components of said bone cement.

### BACKGROUND

Polymethyl methacrylate bone cements stem from the fundamental work of Charnley. Polymethyl methacrylate bone cements are usually composed of a liquid monomer component and a powder component. The monomer component may comprise, for example, the monomer methyl methacrylate and an activator (e.g., N,N-dimethyl-p-toluidine) dissolved therein. The powder component, also referred to as bone cement powder, can comprise one or more polymers producible on the basis of methyl methacrylate and comonomers, such as styrene, methyl acrylate or similar monomers, by polymerization, preferably suspension polymerization. The powder component may further comprise a radiopaque agent and an initiator such as dibenzoyl peroxide. When the powder component is mixed with the monomer component, a plastically deformable dough, the actual bone cement dough, can form as a result of the polymers of the powder component swelling in the methyl methacrylate. When the powder component is mixed with the monomer component, the activator N,N-dimethyl-p-toluidine, for example, reacts with dibenzoyl peroxide to form radicals. The radicals formed can initiate the radical polymerization of the methyl methacrylate. As the polymerization of the methyl methacrylate progresses, the viscosity of the cement dough increases until the cement dough solidifies.

Polymethyl methacrylate bone cements can be mixed in suitable mixing cups with the aid of spatulas by mixing the powder component with the monomer liquid. In the process, air bubbles may be incorporated in the bone cement dough, which may have a negative effect on the mechanical properties of the hardened bone cement.

In order to avoid air inclusions in the bone cement dough, a variety of vacuum cementing systems have been described, of which the following are mentioned by way of example: US6,033,105A, US5,624,184A, US4,671,263A, US4,973,168A, US5,100,241A, WO99/67015A1, EP1020167A2, US5,586,821A, EP1016452A2, DE3640279A1, WO94/26403A1, EP1005901A2, and US5,344,232A.

Cementing systems in which both the cement powder and the monomer liquid are already packed in separate compartments of the mixing systems and are not mixed together in the cementing system until immediately before the cement is applied represent a development in cementing technology. Such closed “fully prepacked mixing systems” are described in patent documents EP0380867B1, EP0796653B1, EP0692229B1, US5997544A, US6709149B1, DE69812726T2 and US5588745A, WO9416951A1, DE19718648A1, EP1741413B1, EP3054880B1, DE102009031178B3, US8662736B2, EP2269718B1, EP2281532B1, and EP3093067B1.

In contrast, patents EP3320869B1 and EP3320870B1 describe a cementing system in which the monomer liquid is mixed with the cement powder by injecting monomer liquid into compacted cement powder.

One advantage of the mixing systems according to EP3320869B1 and EP3320870B1 is that there is no need for any external vacuum source or any mixing elements, such as manually operated stirrers. Our own practical tests have shown that polymethyl methacrylate bone cement dough can be reliably produced using such a device. In these devices, a monomer liquid container arranged in a cartridge is destroyed by advancing a piston, and the monomer liquid is injected into compacted polymethyl methacrylate bone cement powder by applying pressure. The opening of the monomer liquid container and the transferring of the monomer liquid into the polymethyl methacrylate bone cement powder can take place immediately one after the other. The cavity in the cartridge in which the monomer liquid container is arranged and in which the monomer liquid released from the burst monomer liquid container is located is connected by a passage, in a liquid-conducting and gas-conducting manner, to the cavity in the cartridge in which the polymethyl methacrylate bone cement powder is arranged. This means that, once the monomer liquid container has been opened, the monomer liquid should be immediately transferred into the cavity to the polymethyl methacrylate bone cement powder in order to prevent adhesion of the polymethyl methacrylate bone cement powder by monomer vapors or monomer liquid, which can pass unhindered through the passage to the polymethyl methacrylate bone cement. The monomer liquid and also vapors of the monomer swell the polymethyl methacrylate particles, and these swollen particles stick together within a short period of time, which makes subsequent transfer of the monomer liquid difficult or impossible.

In addition, in such conventional systems, such as the mixing systems according to EP3320869B1 and EP3320870B1, a high degree of force is often necessary in order to open a monomer liquid container contained therein and then, by compressing the opened container, to transfer the monomer liquid released therefrom into another compartment of the device that contains a bone cement powder. The force that has to be applied by a user is therefore made up of three components: firstly the force required to break open the monomer liquid container, secondly the force required to compress the resulting fragments or remnants of the container, and thirdly the force required to apply pressure to the released monomer liquid to inject it into a bone cement powder.

### SUMMARY

The invention relates to a device for producing a bone cement, comprising a storage container for receiving a sealed vessel containing a monomer liquid, and a cartridge for mixing bone cement, wherein the cartridge comprises a first chamber for receiving monomer liquid from the storage container and a second chamber for receiving a bone cement powder, and wherein the cartridge further comprises a valve which fluidically connects the first chamber to the second chamber, wherein the device is designed and configured, in a first step, to release a monomer liquid from a sealed vessel within the storage container and to deliver it to the first chamber of the cartridge, in a second step, to keep the monomer liquid within the first chamber, wherein contact of the monomer liquid with the second chamber of the cartridge is avoided, and, in a third step, to deliver the monomer liquid from the first chamber of the cartridge into the second chamber of the cartridge.

One object of the invention is therefore to provide an improved device for storing and mixing polymethyl methacrylate bone cement compared to the devices according to EP3320869B1 and EP3320870B1. One desirable embodiment relates to a "fully prepacked mixing system", in which polymethyl methacrylate bone cement powder and monomer liquid are stored in separate compartments and can be mixed in the device following activation of the latter, without the medical user coming into contact with the polymethyl methacrylate bone cement powder and the monomer liquid. The device can allow the monomer liquid to be temporarily stored in a compartment for a period of a few minutes to one hour after opening the monomer liquid container and transferring the released monomer liquid, without the polymethyl methacrylate bone cement powder being inadvertently able to come into contact with the monomer liquid. Inadvertent contact between the monomer liquid and the polymethyl methacrylate bone cement powder should preferably be prevented. Transferring the monomer liquid and bringing the monomer liquid into contact with bone cement powder should preferably be able to be done only through deliberate action by the user.

Furthermore, it is desirable that as little force as possible is required when discharging the bone cement. The present invention can achieve this by splitting the above-described force to be applied.

### PREFERRED EMBODIMENTS

One object of the present invention is to solve one or more of the problems described above and further problems of the prior art. For example, the invention provides a device for storing and mixing bone cement, which allows easy and reliable mixing of a monomer liquid with a powder component of a PMMA bone cement to produce a ready-to-use PMMA bone cement dough. The devices according to the invention can comprise a plurality of parts which can easily be connected to one another and disconnected from one another again, thereby allowing a reliable and safe transfer of a monomer liquid from one part of the device into the other part of the device.

A first embodiment relates to a device for producing a bone cement, comprising a storage container for receiving a sealed vessel containing a monomer liquid, and a cartridge for mixing bone cement, wherein the cartridge comprises a first chamber for receiving monomer liquid from the storage container and a second chamber for receiving a bone cement powder, and wherein the cartridge further comprises a valve which fluidically connects the first chamber to the second chamber, wherein the device is designed and configured, in a first step, to release a monomer liquid from a sealed vessel within the storage container and to deliver it to the first chamber of the cartridge, in a second step, to keep the monomer liquid within the first chamber, wherein contact of the monomer liquid with the second chamber of the cartridge is avoided, and, in a third step, to deliver the monomer liquid from the first chamber of the cartridge into the second chamber of the cartridge.

A second embodiment relates to a device according to the first embodiment, wherein the storage container and the cartridge are embodied as separate elements, which can be detachably connected to each other.

A third embodiment relates to a device according to the first or second embodiment, wherein the cartridge further comprises a displaceable first piston, which can be connected to the storage container.

A fourth embodiment relates to a device according to any one of the preceding embodiments, wherein the first piston comprises a first passage for delivering a monomer liquid from the storage container to the first chamber of the cartridge.

A fifth embodiment relates to a device according to any one of the preceding embodiments, wherein the cartridge further comprises a displaceable second piston, which is arranged between the first chamber and the second chamber of the cartridge.

A sixth embodiment relates to a device according to any one of the preceding embodiments, wherein the valve is arranged in the second piston.

A seventh embodiment relates to a device according to any one of the preceding embodiments, wherein the second piston comprises a plurality of passages which form a fluidic connection between the valve and the second chamber of the cartridge.

An eighth embodiment relates to a device according to the fifth, sixth or seventh embodiment, wherein the second piston further comprises a fluid-conducting porous layer, which is configured to prevent the ingress of bone cement powder from the second chamber of the cartridge into the valve and/or optionally the passages according to the seventh embodiment.

A ninth embodiment relates to a device according to any one of the preceding embodiments, wherein the valve is designed and configured to keep a monomer liquid entirely within the first chamber when no pressure is applied to the first chamber, and to deliver a monomer liquid to the second chamber when a pressure is applied to the first chamber.

A tenth embodiment relates to a device according to any one of the preceding embodiments, wherein the valve is preferably a valve that can be opened by applying pressure, wherein the valve further preferably comprises a spring and a sealing body mounted on the spring, or comprises a wedged sealing body that can be released by applying pressure, or wherein the valve is a Bunsen valve.

An eleventh embodiment relates to a device according to any one of the preceding embodiments, which is designed and configured to deliver bone cement from the device to the outside as a result of a force being exerted on the first chamber and/or the second chamber.

A twelfth embodiment relates to a device according to any one of the preceding embodiments, further comprising a pressing-out element, wherein the pressing-out element is designed and configured optionally to displace the first piston and/or the second piston.

A thirteenth embodiment relates to a device according to any one of the preceding embodiments, wherein the storage container further comprises an opening element, which is designed and configured to break a glass ampule or to open a film pouch within the storage container.

A fourteenth embodiment relates to a device according to the thirteenth embodiment, wherein the device is designed and configured to deliver a monomer liquid from the storage container to the first chamber of the cartridge by free gravitational flow.

A further aspect relates to the use of a device according to any one of the preceding embodiments for producing a bone cement.

A further aspect relates to a method for producing a bone cement, comprising the following steps of:

(a) providing a device according to any one of the first to fourteenth embodiments,

(b) releasing a monomer liquid from a sealed vessel within the storage container and delivering the monomer liquid to the first chamber of the cartridge,

(c) keeping the monomer liquid within the first chamber of the cartridge, wherein contact of the monomer liquid with the second chamber of the cartridge is avoided,

(d) applying pressure to the monomer liquid in the first chamber and thereby opening the valve and delivering the monomer liquid from the first chamber of the cartridge, through the valve, into the second chamber of the cartridge.

### BRIEF DESCRIPTION OF THE DRAWINGS

FIG. 1 shows a storage container of a device according to the invention;

FIG. 2 shows a cartridge of a device according to the invention;

FIG. 3 shows a device according to the invention, wherein a storage container is brought into contact with a cartridge;

FIG. 4 shows a device according to the invention, wherein a monomer liquid is transferred from the storage container into a first chamber of the cartridge;

FIG. 5 shows a cartridge comprising a first piston, which is closed by a stopper;

FIG. 6 shows a cartridge which is in engagement with a pressing-out rod;

FIG. 7 shows a cartridge in which a first piston is moved toward a second piston by means of a pressing-out rod;

FIG. 8 shows a cartridge in which a first piston is pressed against a second piston by means of a pressing-out rod;

FIG. 9 shows a cartridge containing ready-mixed bone cement, wherein the cartridge is opened by removing a cover; and,

FIG. 10 shows a cartridge containing ready-mixed bone cement, wherein the cartridge comprises a snorkel for dispensing bone cement.

### DETAILED DESCRIPTION OF THE INVENTION

With regard to the embodiments described herein, the elements of which “have”, “contain”, or “comprise” a particular feature (for example, a material), a further embodiment is in principle always contemplated in which the element in question consists solely of the feature, i.e., does not comprise any other constituents. The words “comprise” or “comprising” are used herein synonymously with the words “contain”, “containing”, “have”, or “having”.

“Operatively connected” or “operatively connectable” means herein that two elements in question have a functional relationship to one another. By way of example, a first element may be configured to control or move a second element by such an operative connection. Herein, the term “controlling” also includes blocking or enabling a function, for example allowing or restricting the movement or other function of an element.

When an element is referred to in the singular in an embodiment, an embodiment in which multiple of these elements are present is always also contemplated. The use of a term for an element in the plural in principle also encompasses an embodiment in which only a single such element is present.

Unless otherwise indicated or clearly excluded on the basis of the context, it is possible in principle, and is hereby unambiguously contemplated, that features of different embodiments may also be present in the other embodiments described herein. Likewise, all the features described herein in connection with a method are in principle also considered to be applicable to the products, devices, kits and uses described herein, and vice versa. All these considered combinations are not explicitly listed in all cases, simply in order to keep the description brief. Technical solutions known to be equivalent to the features described herein are also intended in principle to be encompassed by the scope of the invention.

A first embodiment relates to a device for producing a bone cement, comprising a storage container for receiving a sealed vessel containing a monomer liquid, and a cartridge for mixing bone cement, wherein the cartridge comprises a first chamber for receiving monomer liquid from the storage container and a second chamber for receiving a bone cement powder, and wherein the cartridge further comprises a valve which fluidically connects the first chamber to the second chamber, wherein the device is designed and configured, in a first step, to release a monomer liquid from a sealed vessel within the storage container and to deliver it to the first chamber of the cartridge, in a second step. to keep the monomer liquid within the first chamber, wherein contact of the monomer liquid with the second chamber of the cartridge is avoided, and in a third step, to deliver the monomer liquid from the first chamber of the cartridge into the second chamber of the cartridge.

Increased force is often necessary in order to break open a sealed vessel within the device. The devices according to the invention make it possible to decouple the breaking-open of a sealed vessel containing a monomer liquid from the pressing-out of the bone cement mixed in the device. As a result, a first person, e.g. an assistant, can open a sealed vessel containing a monomer liquid within the device and can transfer it into the first chamber of the cartridge, and a second person, e.g. a treating physician, can press the monomer liquid temporarily stored in the first chamber of the cartridge into the second chamber of the cartridge in order thus to produce a ready-to-use bone cement. This bone cement can be pressed out of the cartridge in a further step. As a result, the second person does not have to apply a force to break open the sealed vessel containing monomer liquid and to compress the burst vessel.

The device is preferably suitable for producing a polymethyl methacrylate bone cement. In one embodiment, the device includes a monomer liquid and a bone cement powder.

Polymethyl methacrylate bone cements usually comprise a liquid monomer component, also referred to herein as “monomer liquid”, and a powder component, also referred to herein as “bone cement powder”. The monomer liquid may comprise, for example, the monomer methyl methacrylate and optionally an activator dissolved therein. One example of a suitable activator is N,N-dimethyl-p-toluidine.

The bone cement powder preferably comprises a polymer. Suitable polymers can be produced from methyl methacrylate and comonomers by polymerization. Examples of suitable comonomers include styrene and/or methyl acrylate.

“Bone cement” will be understood herein to mean mixtures of such a monomer liquid and such a bone cement powder. The term “bone cement” includes, in particular, freshly prepared mixtures of these two components, which have a pasty consistency and can harden into a hard polymer.

The device is suitable for receiving a sealed vessel containing a monomer liquid. In some embodiments, the device includes such a vessel. In one embodiment, the vessel is a glass ampule. In one embodiment, the vessel is a film pouch. Preferably, the film pouch comprises a composite material. Suitable composite materials comprise a plastics material and/or a metal, wherein the metal preferably comprises aluminum. Herein, “sealed” will be understood to mean, in particular, that the vessel prevents contact between a monomer liquid contained therein and the surrounding ambient air.

The device according to the invention comprises a storage container and a cartridge. These two elements can be connected to each other. Preferably, the storage container and the cartridge can be detachably connected to each other. This makes it possible to provide the device according to the invention in the form in which the storage container and the cartridge are initially separate from each other. Such provision in the form of two separate components offers advantages in terms of production, sterilization, storage, and transport.

Accordingly, the device may be provided as a kit containing multiple parts. The kit may comprise a storage container as a first part and a cartridge as a second part, as described in detail herein.

The cartridge comprises a first chamber for receiving monomer liquid from the storage container and a second chamber for receiving a bone cement powder.

The first chamber of the cartridge may be used to temporarily store a monomer liquid from the storage container.

The cartridge may be prefilled with a bone cement powder. The second chamber of the cartridge may contain a bone cement powder. Preferably, the second chamber of the cartridge may contain a compacted bone cement powder. This means that the second chamber of the cartridge is substantially completely filled with a bone cement powder, and there are no unfilled cavities in the second chamber of the cartridge. This enables a homogeneous mixing of monomer liquid and bone cement powder, without the need for a mechanical stirring element. In one embodiment, therefore, the device does not comprise a stirring element. One example of a stirring element is a mixing rod comprising one or more mixing blades. In one embodiment, the device is configured to mix a monomer liquid and a bone cement powder simply by injecting the monomer liquid into a compacted bone cement powder. This means that a bone cement is formed from the monomer liquid and the bone cement powder without any mechanical stirring process.

The second chamber preferably comprises a cavity having a volume that exceeds the volume of the monomer liquid to be received by at least 20%, more preferably at least 30%.

The device is configured, in a first step, to release a monomer liquid from a sealed vessel within the storage container and to deliver it to the first chamber of the cartridge. To this end, the storage container may contain a means for opening such a vessel, this also being referred to

herein as an “opening element”. In some embodiments, the storage container further comprises a means for opening a film pouch or a glass ampule within the storage container. One example of a means for opening a film pouch is a spike which is suitable for piercing a film pouch. Such a spike may be made, for example, from a rigid plastics material or from metal. One example of a means for opening a glass ampule is a projection, against which the head of a glass ampule can be pressed to break it open. One means for opening a glass ampule may comprise a flexible housing part, so that the glass ampule can be broken manually from outside by moving and/or deforming the flexible housing part. To this end, the storage container may comprise a flexible housing part and a rigid housing part, wherein the flexible housing part is moved and/or deformed relative to the rigid housing part. One means for opening a glass ampule may also comprise a movably arranged element, such as a piston, which can press the glass ampule against a hard housing part, e.g. a projection, and thereby break it open. Examples of suitable opening elements, in particular for opening a film pouch or a glass ampule, are also disclosed in DE19532015A1, WO9718031A1, EP2404864B1, and WO2010012114A1.

The storage container may further comprise a carrier for receiving a vessel containing a monomer liquid. The carrier is preferably designed and configured to keep the vessel in a defined position within the storage container. The carrier may comprise, for example, a ring-shaped structure which is designed to surround the neck of a glass ampule. Such a ring-shaped structure may be embodied, for example, as an open or closed ring-shaped rib, or may be embodied as an opening in a surface of the carrier.

The carrier may be designed and configured to move the vessel against the opening element. The carrier may be designed and configured to open the vessel in collaboration with the opening element. By way of example, the storage container may comprise a carrier which is movably arranged in the storage container. The carrier may be designed and configured to move the vessel against an opening element, such as a projection, a spike, or a cutting device, in order thus to open the vessel.

The storage container may further comprise a filter, which is designed and configured to retain fragments of the vessel in the storage container. The filter may be embodied, for example, as a screen, in which glass fragments of a broken-open glass ampule can be retained. Such a screen may be embodied as a metal wire mesh or polymer mesh. The screen may also comprise a perforated disk made of metal, glass, ceramic, or plastics material.

The storage container may comprise a first end and a second end. The storage container is preferably designed and configured such as to be able to be connected, preferably detachably connected, to the cartridge at the second end of the storage container.

The storage container may further comprise a nozzle. The nozzle may be designed to deliver monomer liquid from the storage container to the cartridge. The nozzle is preferably arranged at the second end of the storage container. The nozzle may preferably have a conical shape. In one embodiment, the nozzle has a shape that tapers outward. The storage container may thus have a funnel-shaped outlet, which is formed by the nozzle at the second end of the storage container. The nozzle may be arranged centrally at the second end of the storage container. In the case of a cylindrical storage container, this corresponds to the nozzle being arranged in the extension of the longitudinal central axis of the cylinder. The storage container may further comprise a first connecting element, which is designed and configured for connection to a second connecting element of the cartridge. The first connecting element and the second connecting element may form a force-fitting and/or form-fitting connection to each other. Examples of such a connection include a screw closure by means of a thread, or a bayonet closure. The first connecting element and the second connecting element are preferably detachably connected to each other. The first connecting element is preferably attached to the second end of the storage container.

For a secure connection, the first connecting element and/or the second connecting element may further comprise a latching mechanism. In one embodiment, the latching mechanism comprises, for example, one or more movable latching elements which are designed and configured to move radially when the storage container is brought into contact with the cartridge, and to engage in corresponding grooves or recesses on the cartridge. The latching mechanism may preferably be spring-loaded to enable the latching elements to engage in their counterpart.

The latching mechanism may have a release, which is designed and configured to release the latching elements.

In one embodiment, the device according to the invention is designed such that the storage container and the cartridge can be connected to each other in such a way that the respective longitudinal center axes thereof form a common line. This results in an overall linear arrangement of the storage container and the cartridge. In one embodiment, the longitudinal center axis of the storage container forms an angle of 0° to 50°, for example 0° to 40°, 0° to 30°, 0° to 20°, or 0° to 10°, with the longitudinal center axis of the cartridge. In one embodiment, this angle is 0°.

The storage container may further comprise an operating element. Preferably, the operating element is designed and configured to open the vessel. With particular preference, the operating element is designed and configured to open the vessel in collaboration with the opening element of the vessel, which is described herein. The operating element may be embodied, for example, as a grip element which allows a movement of the vessel toward the opening element. By way of example, a user of the device may exert a force on the vessel via the operating element in order to press the vessel against a projection, spike or cutting element of the storage container and thereby open it.

The storage container preferably has a substantially cylindrical shape. In particular, the overall outer surface of the storage container may have a substantially cylindrical shape.

The cartridge comprises a first chamber, a second chamber, and a valve which fluidically connects the first chamber to the second chamber. Preferably, the valve forms the only fluidic connection between the first chamber and the second chamber. This means that monomer liquid must be conducted through the valve in order to pass from the first chamber into the second chamber.

The valve is preferably unidirectional. This means that the valve is designed and configured to allow a monomer liquid to pass only in one direction. Preferably, the valve is designed and configured to conduct monomer liquid from the first chamber into the second chamber of the cartridge. Preferably, the valve is further designed and configured to prevent monomer liquid from flowing from the second chamber into the first chamber of the cartridge.

The valve is preferably a pressure-dependent valve. Such a valve is designed and configured to open when pressure is applied. In one embodiment, the valve is designed and configured to keep a monomer liquid entirely within the first chamber when no pressure is applied to the first chamber, and to deliver a monomer liquid to the second chamber when pressure is applied to the first chamber. The valve may be designed and configured to open above a predetermined limit pressure. According to the invention, such a limit pressure is defined as a difference in pressure applied to the two sides of the valve. This limit pressure, as the difference in pressure between the first chamber and the second chamber of the cartridge, may advantageously be determined by using a manometer at a temperature of 25°C, while the first chamber and the second chamber are filled with ordinary ambient air. By way of example, this limit pressure may be in the range from 0.5 bar to 1.5 bar. In one embodiment, the limit pressure is approximately 1 bar (10^5 Pa), for example 0.8 to 1.2 bar or 0.9 to 1.1 bar. In one embodiment, the limit pressure is approximately 0.5 bar (0.5 x 10^5 Pa), for example 0.3 to 0.7 bar or 0.4 to 0.6 bar.

In one embodiment, the valve is embodied as a check valve. The valve may comprise a spring and a sealing body mounted on the spring.

The valve may comprise a wedged sealing body, which can be released by applying pressure. The sealing body of the valve may be designed and configured to seal the valve when no pressure is applied to the valve and thereby keep it closed. The sealing body of the valve may be designed and configured to release the valve when pressure is applied to the valve, in order to establish a fluidic connection through the valve. The sealing body may have, for example, a spherical, ovoid, or cylindrical shape. The sealing body may comprise or consist of a plastics material, a ceramic, or a metal. The plastics material is preferably a thermoplastic.

The valve may comprise a channel, in which the sealing body is arranged. In one embodiment, the channel has an internal diameter that is at least 1.1 times, preferably at least 2 times, the diameter of the sealing body. This allows the monomer liquid to pass the sealing body when the latter is released from the wedged state.

The valve may be configured to automatically transition from an open state of the valve to a closed state of the valve when the valve transitions from a state in which pressure is applied to a state in which no pressure is applied. As a result, for example, the valve can be opened when pressure is applied to a monomer liquid within the first chamber of the cartridge, in order to transfer the monomer liquid through the valve into the second chamber, and the valve can be closed again after the monomer liquid has been transferred into the second chamber and there is no longer any positive pressure in the first chamber of the cartridge relative to the second chamber of the cartridge.

The device may be designed and configured to hold a defined volume of a monomer liquid. By way of example, the device may include a carrier suitable for commercially available glass ampules or film pouches containing a specific volume of a monomer liquid. In this connection, the first chamber of the cartridge may have an internal volume that exceeds this defined volume of a monomer liquid. By way of example, the first chamber of the cartridge may have an internal volume that exceeds the defined volume of a monomer liquid by at least 20% or preferably by at least 30%.

In one embodiment, the valve is embodied as a Bunsen valve. According to the invention, a Bunsen valve comprises a rubber-elastic material with an opening which is closed when no pressure is applied to the valve and is designed and configured to open when pressure is applied to the valve. By way of example, a Bunsen valve may comprise a rubber disk with a slot. In one embodiment, this slot has a straight, curved, star-shaped, cross-shaped, or horseshoe-shaped shape. In one embodiment, the Bunsen valve is configured to reversibly open and close by means of an elastic restoring force of the rubber-elastic material. In one embodiment, the Bunsen valve comprises slot walls which touch each other in the closed state of a delivery valve and move away from each other when a delivery valve is opened. The slot walls are preferably arranged at right angles to the surface of the rubber-elastic material.

The rubber-elastic material is preferably a polymer, in particular an elastomer. Preferably, the rubber-elastic material comprises or consists of a medically acceptable elastomer. Examples of medically acceptable elastomers include silicone elastomers, thermoplastic elastomers (TPEs), polyisoprene, butyl rubber, nitrile rubber, ethylene propylene diene monomer (EPDM), chloroprene rubber, fluoroelastomers, perfluoroelastomers, and polyacrylate elastomers. Examples of silicone elastomers include polydimethylsiloxane (PDMS) and liquid silicone rubber (LSR).

Examples of thermoplastic elastomers (TPEs) include styrene block copolymers (SBCs), such as styrene-ethylene-butylene-styrene (SEBS), thermoplastic polyurethanes (TPU), and thermoplastic copolyesters (TCE). Examples of polyisoprene include natural rubber and synthetic polyisoprene. Examples of butyl rubber include bromobutyl rubber (BIIR) and chlorobutyl rubber (CIIR). One preferred polyurethane is a polyether urethane. Polyether urethanes can be produced by polyaddition reaction of a polyetherpolyol with a diisocyanate.

In one embodiment, the rubber-elastic material comprises a thermoplastic elastomer. In one embodiment, the rubber-elastic material consists of a thermoplastic elastomer. In one embodiment, the rubber-elastic material comprises a polyether urethane or ethylene propylene diene rubber. In one embodiment, the rubber-elastic material comprises a polyether urethane. In one embodiment, the rubber-elastic material consists of polyether urethane.

In one embodiment, the rubber-elastic material comprises only a single elastomer, i.e., no second elastomer is mixed with the rubber-elastic material. In one embodiment, the rubber-elastic material comprises at least two different materials, for example two different elastomers.

In one embodiment, the rubber-elastic material may have a Young’s modulus of elasticity of 1.2 × 10^7 Pa to 2.1 x × 10^7 Pa, for example 1.3 × 10^7 Pa to 2.0 x × 10^7 Pa, 1.4 × 10^7 Pa to 1.9 x × 10^7 Pa, 1.5 × 10^7 Pa to 1.8 x × 10^7 Pa, or 1.5 × 10^7 Pa to 1.7 x × 10^7 Pa. In another embodiment, the rubber-elastic material may have a Young’s modulus of elasticity of approximately 1.6 × 10^7 Pa. In one embodiment, the rubber-elastic material may have a Young’s modulus of elasticity of 2000 to 2500 psi. The latter corresponds to approximately 1.4 × 10^7 Pa to 1.7 × 10^7 Pa.

The Young’s modulus of elasticity can be determined according to ASTM D412.

The rubber-elastic material is preferably sterilizable using common sterilization processes, i.e., resistant to UV radiation, gamma radiation and treatment with ethylene oxide within the scope of these processes.

The rubber-elastic material is preferably moldable using standard extrusion and/or injection molding processes.

In one embodiment, the rubber-elastic material has a Shore A hardness in the range from 30 to 80. In one embodiment, the rubber-elastic material has a Shore A hardness of 40 to 70, or 50 to 60, for example approximately 55. The Shore A hardness is determined according to ASTM D2240. Elastomers in this range of Shore A hardness have very good restoring force and are very suitable for the manufacture of a rubber-elastic disk or rubber-elastic coating as described herein as part of a delivery valve. Slots in such elastomers close automatically and quickly when the pressure acting thereon drops. In one embodiment, the rubber-elastic material has a Shore A hardness in the range from 75 to 95.

In one embodiment, the cartridge further comprises a displaceable first piston. Preferably, the first piston delimits the first chamber of the cartridge toward the outside. The first piston may be disk-shaped. The first piston may be fitted in the cartridge in a form-fitting manner. By way of example, the first piston may be wedged into a cylindrical interior of the cartridge. In one embodiment, the first piston engages in a latching element, for example a projection or a groove, on the cartridge. In one embodiment, the first piston can be connected to the storage container. In one embodiment, the first piston is displaceable by pressing the storage container against the first piston. This allows a user of the device to first transfer a monomer liquid into the first chamber of the cartridge by bringing the storage container into contact with the cartridge, and then to transfer the monomer liquid from the first chamber of the cartridge into the second chamber of the cartridge by moving the first piston.

The first piston may have an external diameter that exceeds an internal diameter of the cartridge. By way of example, the external diameter of the first piston may be at least 1.1 times the internal diameter of the cartridge. In one embodiment, the external diameter of the first piston is at least 0.1 mm larger than the internal diameter of the cartridge. In one embodiment, the external diameter of the first piston is 0.1 to 1.0 mm larger than the internal diameter of the cartridge. These two measurements each refer to a state of equilibrium of the piston and the cartridge, in which no force is acting thereon that could lead to deformation. In one embodiment, the cartridge has a cylindrical interior, and the internal diameter of the cartridge is defined as the clear width of this interior. In one embodiment, the first piston has a cylindrical shape, and the external diameter of the first piston is defined as the external diameter of this cylindrical shape. Because the first piston is oversized in this way, it can be wedged into the cartridge such that a displacement of the first piston within the cartridge can only take place above a defined force acting on the first piston.

In one embodiment, the first piston comprises a first passage for delivering a monomer liquid from the storage container to the first chamber of the cartridge. The first passage may have, for example, a cylindrical or conical shape. The first passage may be designed and configured to form a connection, preferably a form-fitting connection, with a nozzle of the storage container.

The first passage may have a projection and/or a taper such that the nozzle can only be inserted into the first passage up to a desired position.

The first passage may comprise a closure. In one embodiment, the closure is designed as a stopper. The stopper may be fixedly connected to the first piston or may be designed as a separate part which is not fixedly connected to the first piston. The closure may have a seal. By way of example, the closure may have a circumferential groove, in which a sealing ring is arranged. The stopper may have a conical shape. In one embodiment, the closure has a length sufficient to extend along the entire length of the passage through the first piston. In one embodiment, the closure caps off the first outer piston and/or a second outer surface of the first piston. Preferably, the first outer surface and the second outer surface are arranged at opposite ends of the first piston. Preferably, the first outer surface of the first piston is arranged facing an outer side of the cartridge. Preferably, the second outer surface of the second piston is arranged facing an inner side of the cartridge.

In one embodiment, the device is designed and configured to deliver a monomer liquid from the storage container to the cartridge by free gravitational flow. This means that there is no need to apply pressure to the storage container in order to deliver the monomer liquid from the storage container.

In a further embodiment, the cartridge further comprises a displaceable second piston, which is arranged between the first chamber and the second chamber of the cartridge.

Preferably, the second piston delimits the second chamber of the cartridge toward the outside. The second piston may, for example, be disk-shaped. The second piston may be arranged in the cartridge in a form-fitting manner. By way of example, the second piston may be wedged into a cylindrical interior of the cartridge. In one embodiment, the piston engages in a latching element, for example a projection or a groove, on the cartridge. In one embodiment, the second piston is displaceable by pressing the first piston against the second piston. This allows a user of the device to dispense from the device the bone cement that is mixed in the device.

The second piston may have an external diameter that exceeds an internal diameter of the cartridge. By way of example, the external diameter of the second piston may be at least 1.1 times the internal diameter of the cartridge. In one embodiment, the external diameter of the second piston is at least 0.1 mm larger than the internal diameter of the cartridge. In one embodiment, the external diameter of the second piston is 0.1 to 1.0 mm larger than the internal diameter of the cartridge. These two measurements each refer to a state of equilibrium of the piston and the cartridge, in which no force is acting thereon that could lead to deformation. In one embodiment, the cartridge has a cylindrical interior, and the internal diameter of the cartridge is defined as the clear width of this interior. In one embodiment, the second piston has a cylindrical shape, and the external diameter of the second piston is defined as the diameter of this cylindrical shape. Because the second piston is oversized in this way, it can be wedged into the cartridge such that a displacement of the second piston within the cartridge can only take place above a defined force acting on the second piston.

In one embodiment, the first piston and the second piston each have the same external diameter. In one embodiment, the first piston and the second piston have substantially the same external shape. This refers to the basic shape of the first piston and the second piston; in other words, elements contained therein, such as a passage or a valve, are not taken into consideration. By way of example, the first piston and the second piston may each have a substantially disk-shaped external shape.

Preferably, the valve which fluidically connects the first chamber of the cartridge to the second chamber of the cartridge is arranged in the second piston.

The second piston may further comprise a plurality of passages. These passages may form a fluidic connection between the valve and the second chamber of the cartridge. These passages may be designed and configured to conduct a monomer liquid, which is delivered from the first chamber into the second chamber, into the second chamber at different positions. This allows the monomer liquid to be better distributed within the second chamber and thus mix more evenly with a bone cement powder contained therein.

In one embodiment, the second piston further comprises a fluid-conducting porous layer. This layer is preferably configured to prevent the ingress of bone cement powder from the second chamber of the cartridge into the valve and/or the passages of the second piston. This can prevent the valve and/or the passages from becoming blocked. The porous layer may be, for example, a metal wire mesh or a mesh made of a plastics material. The porous layer may also be a disk which is made of metal or plastics material, and which has suitably dimensioned holes. Furthermore, the porous layer may comprise open-pore foams, such as Porex® foam materials. Preferably, the porous layer may be made of a material which is not attacked by the monomer liquid used in the device.

In one embodiment, the device is designed and configured to deliver bone cement from the device to the outside by exerting a force on the first chamber and/or the second chamber. To this end, the cartridge may comprise a movable first piston and a movable second piston. By exerting pressure on the first piston, the latter can first be moved toward the second piston, so that the second piston is also moved by the first piston, thus causing pressure to build up in the second chamber. As a result, the bone cement can be pressed out of the second chamber of the cartridge.

In one embodiment, the device further comprises a pressing-out element. The pressing-out element is preferably designed and configured to displace the first piston and/or the second piston. The pressing-out element may comprise a rod which is designed and configured to transmit a force to the first piston. In one embodiment, the pressing-out element comprises a threaded rod. The cartridge may comprise a connecting means for mechanical connection to the pressing-out element. The connecting means may, for example, have a thread. In one embodiment, the connecting means is designed and configured to form a rotationally fixed connection with the pressing-out element. The pressing-out element is preferably designed and configured to press bone cement out of the second chamber of the cartridge.

The pressing-out element may comprise a threaded rod having an external thread. In one embodiment, a pitch of the external thread is at least 1 mm, preferably at least 3 mm and/or at most 7 mm, preferably at most 5 mm. This has proven to be particularly effective in tests. In one embodiment, the external diameter of the external thread of the threaded rod is at least 12 mm and/or at most 17 mm. The external diameter means the maximum diameter measured between the two maximum peaks of the thread turn, i.e., the diameter of an imaginary circular cylinder surrounding the external thread. In particular, the external diameter is at least 13.5 mm and/or at most 15 mm. In one embodiment, the external diameter is approximately or exactly 14 mm. An external diameter of less than 12 mm is not suitable to withstand the forces or moments that occur. An external diameter that is too large increases friction and thus the force required for discharge. In one embodiment, the internal thread of the adapter unit contains at least 2 thread turns, preferably at least or exactly 4 thread turns, and/or at most 6 thread turns, preferably at most 5 thread turns. In one embodiment, the thread of the threaded rod is a trapezoidal thread. This has proven to be particularly suitable. In one embodiment, the external thread of the threaded rod has a thread depth of at least 1 mm and/or at most 3 mm or 4 mm.

The cartridge may further comprise a receptacle for connection to a pressing-out element. The pressing-out element may comprise a round disk which is designed and configured to engage in a receptacle of the cartridge. The cartridge may further comprise a cartridge head. The above-described receptacle may be arranged on this cartridge head. As set out above, the pressing-out element may have a thread. The cartridge head may have a thread which is designed and configured to engage in a thread of the pressing-out element. In one embodiment, the device is designed and configured to push the first piston into the cartridge, with the aid of the pressing-out element, to deliver a monomer liquid from the first chamber of the cartridge, through the valve, into the second chamber of the cartridge.

In one embodiment, the storage container further comprises an opening element, which is designed and configured to break a glass ampule or to open a film pouch within the storage container.

In one embodiment, the storage container further comprises an operating element for controlling the opening element. Preferably, the operating element is operatively connected to the opening element. The storage container may further comprise a safety device, which protects the operating element against inadvertent actuation. Such a safety device may comprise, for example, a clamp or a latching element.

The storage container may comprise a first end and a second end. The operating element may be arranged at the first end of the storage container.

In one embodiment, the operating element is movable toward the second end of the storage container. As a result, a vessel containing a monomer liquid, for example a glass ampule or a film pouch, can be brought into contact with an opening element in order to open the vessel. The opening element may comprise, for example, a projection, a spike, or a blade. In one embodiment, the storage container comprises an opening element which comprises a projection, which is designed and configured to break open the neck of a glass ampule by pressing it against the projection to open the glass ampule.

The storage container may comprise a carrier, which is designed and configured to receive a sealed vessel containing a monomer liquid. In one embodiment, the operating element is designed and configured to move the carrier toward an opening element.

In one embodiment, the storage container is of multi-part design. The storage container may comprise a flexible sub-element and a rigid sub-element. The storage container may be designed and configured to open a vessel containing a monomer liquid by exerting a force on the flexible sub-element such that the flexible sub-element moves relative to the rigid sub-element or the flexible sub-element deforms. One example of this functional principle is described in detail in EP2404864B1.

A further aspect relates to the use of a device described herein for producing a bone cement. The device can be used as described herein to produce bone cement from a monomer liquid and a bone cement powder. In this case, the monomer liquid can preferably be released within the device and mixed with the bone cement powder to form a bone cement.

The device may further comprise a snorkel for dispensing bone cement. The snorkel can preferably be connected to the cartridge. In one embodiment, the snorkel can be detachably connected to the cartridge. The cartridge may have a thread for connection to such a snorkel. The cartridge may have an outlet which is fluidically connected to the second chamber of the cartridge. The snorkel may preferably be connectable to such an outlet.

The cartridge may further comprise a closure. The closure may be provided with a thread. The closure may be detachably connectable to the cartridge. In one embodiment, the closure is arranged at the second end of the cartridge. In one embodiment, the closure can be connected, preferably can be detachably connected, to an outlet described above.

In one embodiment, a kit is provided which comprises a storage container described herein and a cartridge described herein, the kit further comprising a closure and a snorkel, the closure and the snorkel each being interchangeably connectable to an outlet of the cartridge.

A further aspect relates to a method for producing a bone cement, comprising the steps of:

(a) providing a device described herein;

(b) releasing a monomer liquid from a sealed vessel within the storage container and delivering the monomer liquid to the first chamber of the cartridge;

(c) keeping the monomer liquid within the first chamber of the cartridge, wherein contact of the monomer liquid with the second chamber of the cartridge is avoided; and,

(d) applying pressure to the monomer liquid in the first chamber and thereby opening the valve and delivering the monomer liquid from the first chamber of the cartridge, through the valve, into the second chamber of the cartridge.

The device used in this method preferably comprises a storage container for receiving a sealed vessel containing a monomer liquid, as well as a cartridge comprising a first chamber and a second chamber, wherein the second chamber is designed and configured to receive a bone cement powder, and wherein the first chamber is fluidically connected to the second chamber via a valve.

In one embodiment, the monomer liquid is kept within the first chamber of the cartridge for a period of time longer than 1 minute and shorter than 1 hour.

The method may further comprise mixing a monomer liquid, a bone cement powder, and an antibiotic. Examples of suitable antibiotics include gentamicin, vancomycin and daptomycin.

The method may further comprise producing a bone cement by mixing the monomer liquid with a bone cement powder. This mixing process preferably takes place within the second chamber of the cartridge. The method may further comprise delivering a bone cement out of the device. The bone cement may be delivered in vitro, or delivery may take place directly to a patient, wherein the bone cement is brought into direct contact with patient tissue.

The method may further comprise producing a spacer from a bone cement produced in the device according to the invention. To this end, the bone cement can first be produced in the device and then transferred to a casting mold for a spacer.

A further aspect of the invention relates to a treatment method, wherein a bone cement produced using a device described herein is delivered to a patient. The treatment method may comprise implanting an artificial joint or a spacer.

### EXAMPLES

The invention will be further illustrated below on the basis of examples, but these are not to be understood as limiting. It will be apparent to a person skilled in the art that other equivalent means may be used similarly in place of the features described here.

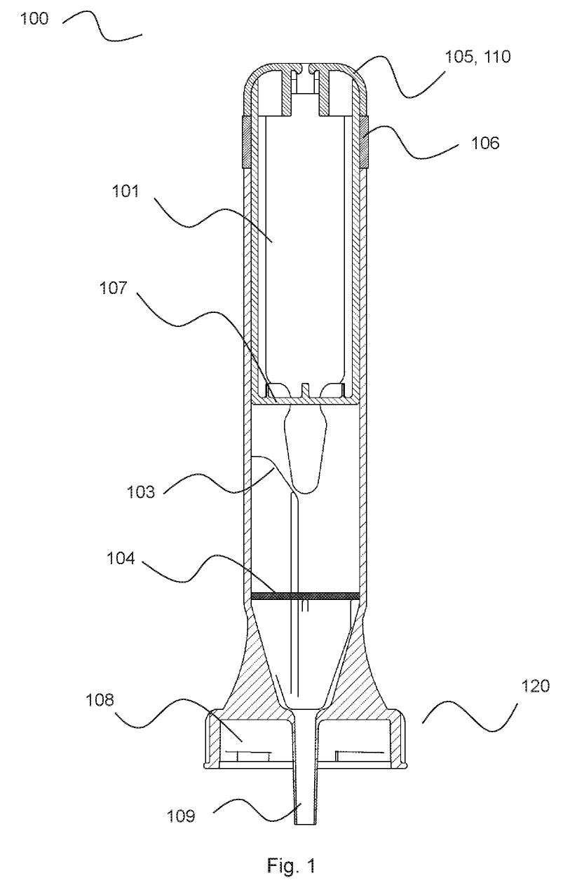

FIG. 1 shows a storage container 100 of a device according to the invention. The storage container 100 comprises a housing having a substantially cylindrical shape. The storage container 100 comprises a first end 110 and an opposite second end 120. The storage container 100 further comprises a carrier 107. The carrier 107 is designed and configured to hold a vessel 101 containing a monomer liquid 102. In the example shown here, the vessel 101 is embodied as a glass ampule. The glass ampule is arranged in the carrier 107. In the arrangement shown here, the neck of the glass ampule protrudes from the carrier 107. The carrier 107 is movably arranged in the storage container 100 in such a way as to enable a movement of the vessel 101 to open it. This movement takes place by way of an operating element 105, which is operatively connected to the carrier 107. The operating element 105 is arranged at the first end 110 of the storage container. By moving the operating element 105, the carrier 107 and thus the vessel 101 arranged therein can be moved toward the second end of the storage container. As a result, the vessel 101 can be brought into contact with an opening element 103 to open the vessel. Here, the opening element 103 is embodied as a projection, which is arranged within the storage container 100. The vessel 101 can thus be opened by moving the operating element 105. In the example shown here, the neck of the glass ampule 101 can be broken away from the ampule 101 by pressing it against the projection 103, to open the ampule. The storage container 100 further comprises a filter 104 for retaining fragments of an opened vessel. These could be, for example, fragments of glass from an opened glass ampule or portions of an opened film pouch. The storage container 100 further comprises, at its second end 120, a first connecting element 108 which is designed and configured to connect the storage container 100 to a cartridge 200 in a liquid-tight manner. The storage container 100 further comprises, at its second end 120, a nozzle 109 which is designed and configured to deliver a liquid to a cartridge 200. The storage container 100 comprises an interior which tapers toward the nozzle 109 at the second end 120. The embodiment of the storage container 100 shown here further comprises a safety device 106, which protects the operating element 105 against inadvertent actuation and movement. Here, the safety device 106 is embodied as a safety clamp, which partially surrounds the storage container 100 and limits the movement of the operating element 105. The safety device 106 is detachably connected to the storage container 100. Once the safety device 106 has been removed, the operating element 105 can be moved to open the vessel 101 by means of the opening element 103.

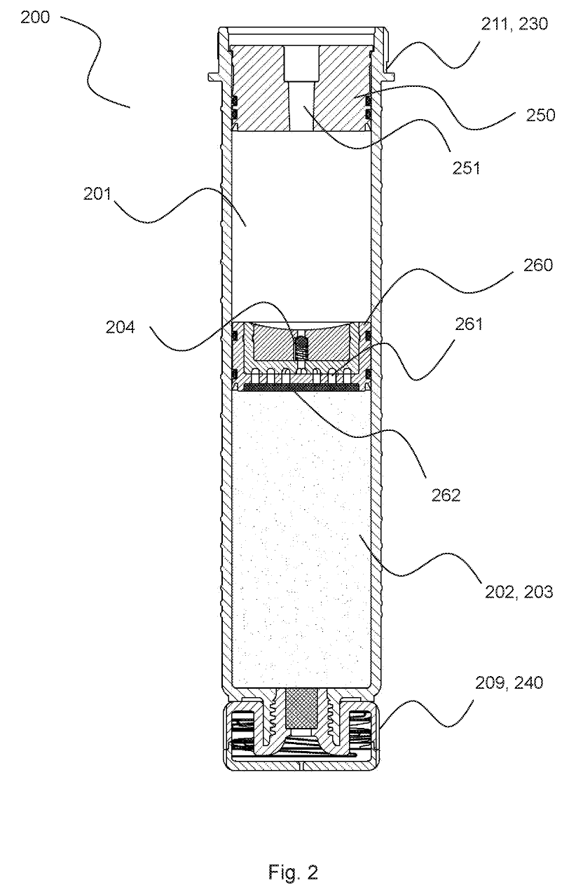

FIG. 2 shows a cartridge 200 of a device according to the invention. The cartridge 200 comprises a first end 230 and a second end 240. The cartridge 200 comprises a first chamber 201 and a second chamber 202. In the embodiment shown here, the first chamber 201 is arranged at the first end 230, and the second chamber 202 is arranged at the second end 240. The first chamber 201 is designed and configured to receive a monomer liquid from a storage container according to the invention. A first piston 250 is arranged at the first end 230. The first piston 250 is movably arranged in the cartridge 200 and delimits the first chamber 201 of the cartridge toward the outside. The first piston 250 comprises a first passage 251. The first passage 251 is designed and configured to form a form-fitting, fluidic connection with the nozzle 109.

The second chamber 202 is designed and configured to receive a bone cement powder 203. In the example shown here, a bone cement powder 203 is arranged in the second chamber 202. A second piston 260 is movably arranged in the cartridge 200 between the first chamber 201 and the second chamber 202 of the cartridge. The second piston 260 comprises a valve 204 which forms a fluidic connection between the first chamber 201 and the second chamber 202 of the cartridge. The valve 204 is unidirectional. This means that a monomer liquid can flow from the first chamber 201 through the valve 204 into the second chamber 202, but no monomer liquid or bone cement powder can pass from the second chamber 202 into the first chamber 201. The valve 204 is pressure-dependent, so that a monomer liquid can be kept in the first chamber 201 when no pressure is applied and can be delivered from the first chamber 201 into the second chamber 202 when pressure is applied.

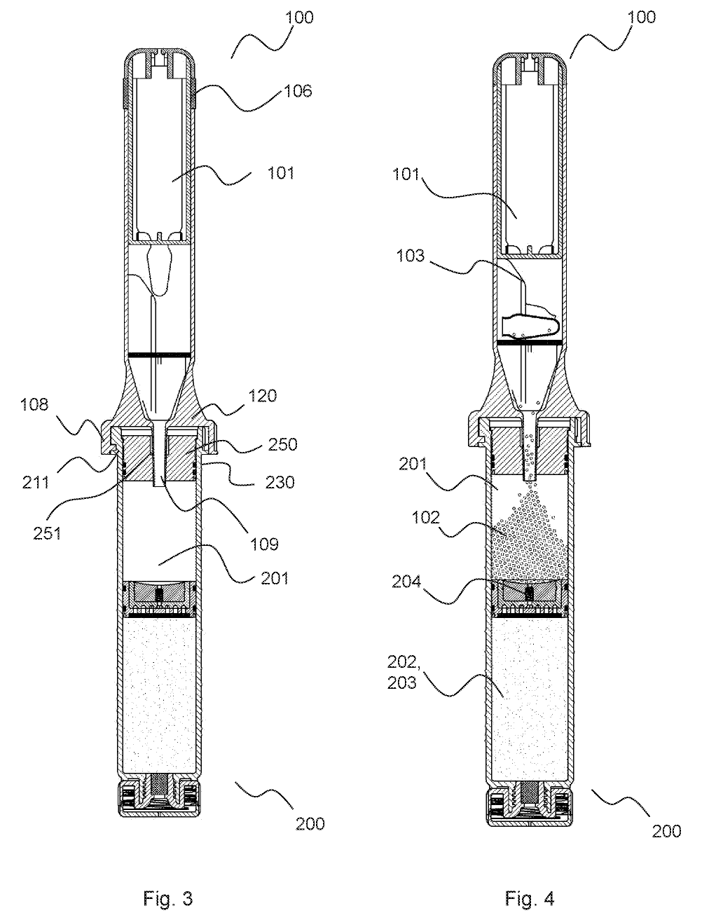

FIG. 3 shows a device according to the invention, wherein a storage container 100 is brought into contact with a cartridge 200. The second end 120 of the storage container is directly connected to the first end 230 of the cartridge. To this end, the storage container 101 comprises a first connecting element 108, which engages in a second connecting element 211 of the cartridge 200. In the embodiment shown here, the first connecting element 108 and the second connecting element 211 together form a form-fitting connection in the manner of a bayonet lock. The nozzle 109 is inserted into a first passage 251 of the first piston in order to establish a fluidic connection between the storage container 100 and the first chamber 201 of the cartridge. A safety device 106 prevents the movement of the operating element 105. In this way, inadvertent opening of the vessel 101 can be prevented.

FIG. 4 shows a device according to the invention, wherein a monomer liquid 102 is transferred from the storage container 100 into a first chamber 201 of the cartridge 200. The vessel 101 has been opened with the aid of an opening element 103 to release the monomer liquid 102 initially within the storage container 100 and then deliver it to the first chamber 201 of the cartridge. The delivery of the monomer liquid 102 to the first chamber 201 can take place without any application of pressure, for example by free gravitational flow. In the state of the device shown here, the monomer liquid 102 is kept in the first chamber of the cartridge 201. Here, no pressure is being applied to the monomer liquid 102. Contact of the monomer liquid with the second chamber 202 of the cartridge 200 is thus avoided, since the valve 204 is closed when no pressure is applied to the monomer liquid 102. This allows a controlled delivery of the monomer liquid from the first chamber 201 to the second chamber 202 of the cartridge and storage of the monomer liquid within the first chamber 201. The monomer liquid 102 can be kept in the first chamber 201 for a period of a few minutes, for example, without any chemical reaction taking place between the monomer liquid 102 and components of the outside air or bone cement powder.

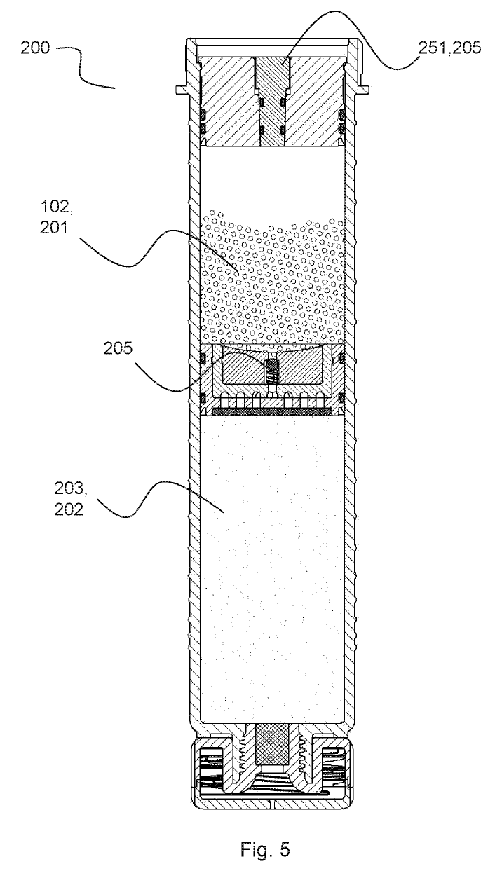

FIG. 5 shows a cartridge 200 comprising a first piston 250 which is closed by a stopper 205. The state of the cartridge 200 shown here can be achieved once a vessel containing a monomer liquid in the storage container has been opened, as shown in FIG. 3 for example, and the monomer liquid has been delivered to the first chamber 201, as shown in FIG. 4 for example. A monomer liquid 102 is kept in the first chamber 201. The valve 205 is closed since no pressure is being applied to the monomer liquid. The first passage 251, which is arranged in the first piston 250, is closed by a stopper 205 to prevent the monomer liquid 102 from leaving the first chamber 201. The monomer liquid 102 can thus be kept in the first chamber 201. This allows the user of the device to temporarily store the monomer liquid and to keep it ready for mixing with a bone cement powder 203, which is arranged in the second chamber 202, until a desired point in time. In the example shown here, the stopper 205 comprises circumferential rubber seals to delimit the first chamber 201 toward the outside in a liquid-tight manner.

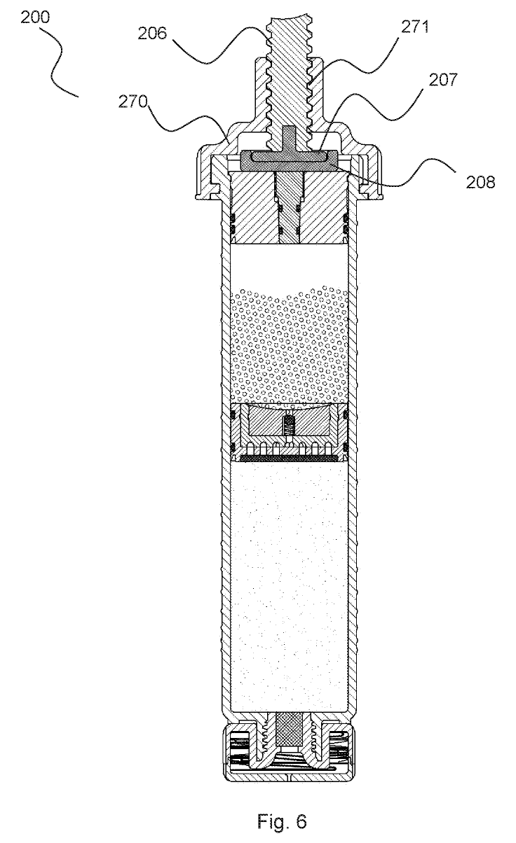

FIG. 6 shows a cartridge 200 which is in engagement with a pressing-out rod 206. The pressing-out rod 206 comprises a round disk 207, which engages in a receptacle 208 arranged on the first piston 250. The cartridge 200 comprises a cartridge head 270 at the first end 230 of the cartridge. The cartridge head 270 has an internal thread 271. The pressing-out rod 206 can engage in the thread 271 to exert a force on the first piston 250. Therefore, by means of the pressing-out rod 206, the first piston 250 can be pushed into the cartridge 200 to deliver a monomer liquid 102 from the first chamber of the cartridge 201, through the valve 204, into the second chamber 202 of the cartridge.

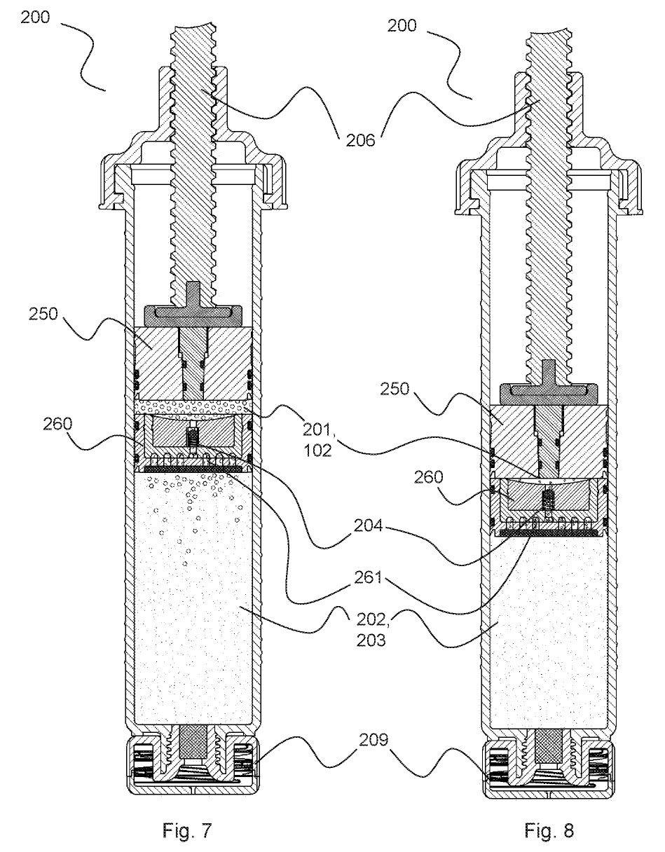

FIG. 7 shows a cartridge 200 in which a first piston 250 is moved toward a second piston 260 by means of a pressing-out rod 206. The movement of the first piston 250 leads to a reduction in the internal volume of the first chamber 201, so that pressure is applied to the monomer liquid 102 contained therein. As a result, the pressure-dependent valve 204 opens so that the monomer liquid 102 is delivered from the first chamber 201 to the second chamber 202 of the cartridge. In doing so, the monomer liquid 102 is conducted through the valve 204, a second passage 261, and a porous layer 262. The porous layer serves as a filter since it is permeable to air and the monomer liquid, but impermeable to the bone cement powder. The monomer liquid 102 thus enters into contact with a bone cement powder 203 within the second chamber 202, wherein a bone cement is formed from the monomer liquid and the bone cement powder.

FIG. 8 shows a cartridge in which a first piston 250 is pressed against a second piston 260 by means of a pressing-out rod 206. As a result, the internal volume of the first chamber 201 of the cartridge is reduced to a minimum volume, which is also referred to as the dead volume. The monomer liquid 102 has been substantially entirely delivered from the first chamber 201 to the second chamber 202 of the cartridge. By moving the first piston 250 further, the second piston 260 will also be moved toward the second end of the cartridge. As a result, pressure is applied to the contents of the second chamber 202 of the cartridge. The second chamber 202 is delimited toward the outside by a closure 209. In the present case, the closure 209 is embodied as a screw cap. The cartridge comprises a thread at the second end 240 for connection to this screw cap.

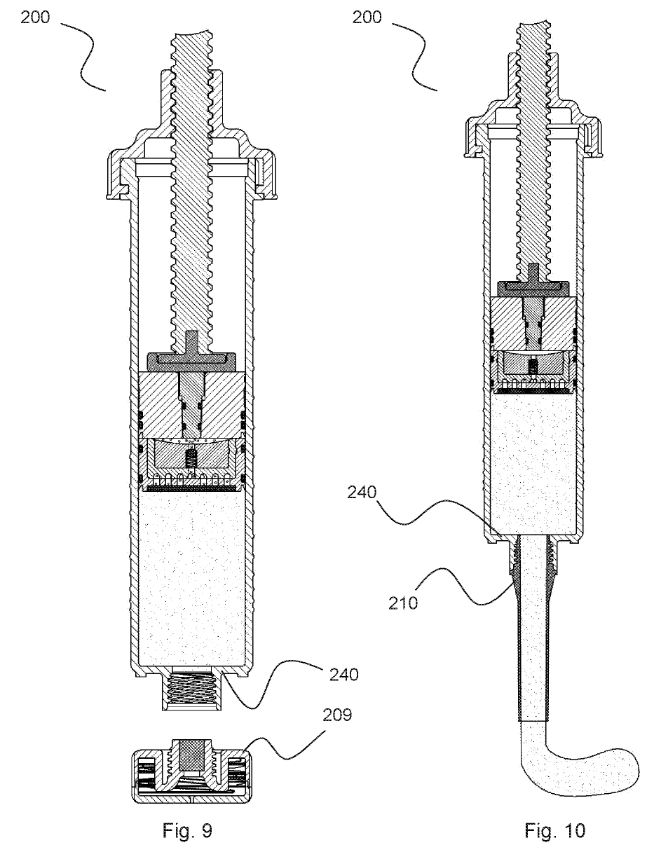

FIG. 9 shows a cartridge 200 containing ready-mixed bone cement, wherein the cartridge is opened by removing a closure 209. This enables the bone cement to be dispensed from the cartridge.

FIG. 10 shows a cartridge containing ready-mixed bone cement, wherein the cartridge comprises a snorkel 210 for dispensing bone cement. In the present case, the cartridge comprises a thread at the second end 240, which enables the snorkel 210 to be screwed onto the cartridge.

### LIST OF REFERENCE SIGNS

100 Storage container

101 Vessel

102 Monomer liquid

103 Opening element

104 Filter

105 Operating element

106 Safety device

107 Carrier

108 First connecting element

109 Nozzle

110 First end of the storage container

120 Second end of the storage container

200 Cartridge

201 First chamber of the cartridge

202 Second chamber of the cartridge

203 Bone cement powder

204 Valve

205 Stopper

206 Pressing-out rod

207 Round disk

208 Receptacle for round disk 207

209 Closure

210 Snorkel

211 Second connecting element

230 First end of the cartridge

240 Second end of the cartridge

250 First piston

251 first passage

260 Second piston

261 Second passage

262 Porous layer

270 Cartridge head

271 Thread of the cartridge head

What is claimed is:

1. A device for producing a bone cement, comprising a storage container for receiving a sealed vessel containing a monomer liquid, and a cartridge for mixing bone cement, wherein the cartridge comprises a first chamber for receiving monomer liquid from the storage container and a second chamber for receiving a bone cement powder, and wherein the cartridge further comprises a valve which fluidically connects the first chamber to the second chamber,
wherein the device is designed and configured to perform the following steps:
in a first step, to release a monomer liquid from a sealed vessel within the storage container and to deliver it to the first chamber of the cartridge;
in a second step, to keep the monomer liquid within the first chamber, wherein contact of the monomer liquid with the second chamber of the cartridge is avoided; and,
in a third step, to deliver the monomer liquid from the first chamber of the cartridge into the second chamber of the cartridge.

2. The device according to claim 1, wherein the storage container and the cartridge are embodied as separate elements, which can be detachably connected to each other.

3. The device according to claim 1, wherein the cartridge further comprises a displaceable first piston, which can be connected to the storage container.

4. The device according to claim 3, wherein the first piston comprises a first passage for delivering a monomer liquid from the storage container to the first chamber of the cartridge.

5. The device according to claim 1, wherein the cartridge further comprises a displaceable second piston, which is arranged between the first chamber and the second chamber of the cartridge.

6. The device according to claim 5, wherein the valve is arranged in the second piston.

7. The device according to claim 6, wherein the second piston comprises a plurality of passages which form a fluidic connection between the valve and the second chamber of the cartridge.

8. The device according to claim 5, wherein the second piston further comprises a fluid-conducting porous layer, which is configured to prevent the ingress of bone cement powder from the second chamber of the cartridge into the valve and/or optionally the passages which form a fluidic connection between the valve and the second chamber of the cartridge.

9. The device according to claim 1, wherein the valve is designed and configured to keep a monomer liquid entirely within the first chamber when no pressure is applied to the first chamber, and to deliver a monomer liquid to the second chamber when pressure is applied to the first chamber.

10. The device according to claim 1, wherein the valve is preferably a valve that can be opened by applying pressure, wherein the valve further preferably comprises a spring and a sealing body mounted on the spring, or comprises a wedged sealing body that can be released by applying pressure, or wherein the valve is a Bunsen valve.

11. The device according to claim 1, which is designed and configured to deliver bone cement from the device to the outside as a result of a force being exerted on the first chamber and/or the second chamber.

12. The device according to claim 1, further comprising a pressing-out element, wherein the pressing-out element is designed and configured optionally to displace the first piston and/or the second piston.

13. The device according to claim 1, wherein the storage container further comprises an opening element, which is designed and configured to break a glass ampule or to open a film pouch within the storage container.

14. The device according to claim 1, wherein the device is designed and configured to deliver a monomer liquid from the storage container to the first chamber of the cartridge by free gravitational flow.

15. A use of a device according to claim 1 for producing a bone cement.

16. A method for producing a bone cement, comprising the following steps (a) to (d) of:
(a) providing a device according to claim 1;
(b) releasing a monomer liquid from a sealed vessel within the storage container and delivering the monomer liquid to the first chamber of the cartridge;
(c) keeping the monomer liquid within the first chamber of the cartridge, wherein contact of the monomer liquid with the second chamber of the cartridge is avoided; and,
(d) applying pressure to the monomer liquid in the first chamber and thereby opening the valve and delivering the monomer liquid from the first chamber of the cartridge, through the valve, into the second chamber of the cartridge.

17. The device according to claim 2, wherein the cartridge further comprises a displaceable first piston, which can be connected to the storage container.

18. The device according to claim 6, wherein the second piston further comprises a fluid-conducting porous layer, which is configured to prevent the ingress of bone cement powder from the second chamber of the cartridge into the valve and/or optionally the passages which form a fluidic connection between the valve and the second chamber of the cartridge.

19. The device according to claim 7, wherein the second piston further comprises a fluid-conducting porous layer, which is configured to prevent the ingress of bone cement powder from the second chamber of the cartridge into the valve and/or optionally the passages which form a fluidic connection between the valve and the second chamber of the cartridge.

20. The device according to claim 2, wherein the valve is designed and configured to keep a monomer liquid entirely within the first chamber when no pressure is applied to the first chamber, and to deliver a monomer liquid to the second chamber when pressure is applied to the first chamber.
